(19) United States
(12) Patent Application Publication
(10) Pub. No.: US 2016/0067461 A1
(43) Pub. Date: Mar. 10, 2016

(54) Controlled Burst Flexible Medical Balloon with Axially Constant Radial Pressure

(71) Applicant: Daniel Gelbart, Vancouver (CA)

(72) Inventor: Daniel Gelbart, Vancouver (CA)

(21) Appl. No.: 14/845,162

(22) Filed: Sep. 3, 2015

Related U.S. Application Data
(60) Provisional application No. 62/067,706, filed on Oct. 23, 2014.
(60) Provisional application No. 62/048,498, filed on Sep. 10, 2014.

(51) Int. Cl. A61M 25/10

(52) U.S. Cl. CPC A61M 25/1018; A61M 2025/1043

(57) ABSTRACT

A high pressure balloon uses a shallow helical groove embossed in the balloon wall with axially constant radial pressure. A filament made from a high tensile strength material with low compliance is wrapped in the embossed groove to prevent internal pressure within the balloon from flattening the groove. The balloon is designed to split longitudinally in case of excessive pressure.

### TECHNICAL FIELD

The invention relates to balloons, or balloon catheters, for use in medical procedures such as stent deployment, angioplasty, heart valve placement, aortal graft placement and other procedures requiring high-pressure balloons.

### BACKGROUND

Medical balloons, particularly, those used in the vascular system, should satisfy several opposing requirements. Since balloons are typically introduced through small diameter sheaths, it is desirable to make balloons from very thin material to facilitate folding the balloons to small diameters. However, such balloons are often operated at high pressures, typically from 10 to 20 atmospheres, which requires the balloons to be made from high tensile strength materials such as Nylon12, polyurethane, PET or PEBAX. Balloon materials should have a limited ability to stretch, as the shape of the balloon should follow the designed shape and not be affected by the resistance it encounters while expanding.

While meeting all these requirements, a balloon should have axially constant radial pressure along the longitudinal length of the balloon in a fully-inflated state and still be flexible in a deflated (folded) state. Flexibility when folded is referred to as “crossing profile” or “trackability”. This property allows a balloon to follow a tortuous vascular path (e.g. following a guidewire through vascular passages). Trackability can be achieved by using very thin materials for the balloon wall with proper folding. For this reason, medical balloons are typically made of material having thickness in the range of 10 to 30 microns. A balloon should also have a low straightening force and, therefore, axially constant radial pressure when fully inflated, as the balloon may be placed in a bent part of an artery or vein when inflated. Forcing the artery or vein to straighten, even momentarily, can cause trauma and can lead to stenosis.

Unfortunately, the same qualities that have been exploited to allow balloons to have good trackability and well defined shape tend to give the balloons a larger difference of pressure between the center and ends of the balloon when inflated. This is illustrated in FIG. 1 which is a schematic view of a prior art balloon 1. Balloon 1 has a very thin wall 4. Balloon 1 has an inflation tube 2 and a guidewire passage 3. Balloon 1 is normally straight. If one attempts to bend balloon 1 to form a curve the portion 4′ of wall 4 on the inside of the curve tends to become pushed in or wrinkled because the wall material has a very limited elastic range, typical of the high tensile strength materials used. For this to happen, the volume of the balloon would need to decrease. Since such balloons are typically inflated by a non-compressible fluid (e.g. saline) using a positive displacement piston pump, any change that would reduce the volume of the balloon is resisted by increased fluid pressure within the balloon. As a result, the balloon resists bending with a straightening force which tries to keep the balloon straight with a circular cross section. For a balloon to have good bending flexibility and the radial pressure should be nearly constant when inflated, its volume should not change while the balloon is being bent. This requires the wall on the inner radius of the bend to compress and the outer wall to stretch while keeping the cross-section circular.

Another important requirement balloons should satisfy is to split longitudinally in case of excessive pressure. When a balloon splits longitudinally it can be pulled out easily through the blood vessels. If it splits circumferentially it can potentially “snag” the blood vessel walls and severely damage the walls when pulled out.

Prior art attempts to reduce the difference in pressure along the axis of medical balloons can be grouped into five categories, as illustrated in FIGS. 2A to 2E. The simplest idea is to make the balloon from a softer material that is more elastic (i.e. a material that has a smaller modulus of elasticity). This is illustrated symbolically in FIG. 2A. The problem is that the balloon will not have a controlled shape. It will expand according to the resistance it encounters and also will elongate significantly with inflation, as the longitudinal stress is a significant portion of the hoop (circumferential) stress.

Balloons are classified as “compliant” (elastic) or “non-compliant” (inelastic), which are relative terms. Any material has an elastic range. When this range is exceeded material can either break or undergo a plastic deformation. Non-compliant balloons typically have an elastic range of under 20%, and even compliant balloons used in vascular procedures have a limited elastic range, typically 20%-40%.

FIG. 2B shows an example of a compliant balloon wrapped with a less compliant filament 5. This general structure is disclosed in several U.S. Pat. Nos. and patent applications including 2014/0172066, 2010/0318029, 2007/0112370, 2004/006359, 8,672,990, 8,349,237, 8,221,351, 8,105,275, 8,002,744, 7,914,487, 6,824,553, 6,773,447, 5,772,681, 4,498,473, 8,486,014, 2010/0286760, 2011/0029064, 7,803,180, 6,878,162, 2002/0103529, 5,449,373, 2014/0135891, 5,569,220, 5,171,297, 6,245,040, 6,626,861, 7,001,420, 6,641,603, and others. Such a balloon is usable only at relatively low inflation pressures as the more elastic materials have a significantly lower tensile strength. The wound filament does not increase the longitudinal strength. If the filament is wound in a two-dimensional mesh pattern, to increase longitudinal strength, the material becomes non-compliant and stronger, but loses its flexibility and increases the difference in radial pressure along the longitudinal axis.

FIG. 2C shows another example balloon in which deep grooves 6 are formed in the wall of a non-compliant balloon to increase flexibility. Balloons having this general configuration are described in U.S. Pat. Nos. 8,257,418 and 5,545,132. While grooves 6 may improve the crossing profile and blood flow during deployment, such embossing disappears when the balloon is fully pressurized as the thin wall (10 to 30 μm typically) cannot keep the embossed shape against the high pressure, typically 10 to 20 atm, which tries to maximize balloon volume. This fact is clearly stated in both these patents (page 2 lines 45-51 in 8257418 and page 5 lines 20-36 in U.S. Pat. No. 5,545,132). Shallow embossing is also used in a commercial product, the Rival TM balloon made by Bard Vascular (Tempe, Az.), but this embossing also disappears when the balloon is fully pressurized. The embossing is provided to improve trackability when the balloon is deflated. Deep grooves were achieved in U.S. Pat. No. 4,762,130, wherein the grooves themselves were achieved by wrapping an inflatable helical balloon around a catheter

FIG. 2D shows a non-compliant balloon divided into longitudinal segments. U.S. Pat. Nos. 6,048,350, 6,776,771, 7,658,744, 7,740,609, and 6,022,359 show examples of this construction. Expanding a stent or artery with such a balloon leads to an imprint in the shape of the segments which is highly undesirable. The transition between segments is wide, as a tapered section is needed at the ends of each segment. Without a tapered section the stress is too high.

FIG. 2E shows another prior art balloon. The balloon is divided longitudinally into several smaller balloons. U.S. Pat. No. 8,758,386 provides an example of this construction. This balloon can have good performance but it is undesirably expensive to manufacture.

There remains a need for medical balloons that provide axially constant radial pressure along the length of the balloon and yet are cost effective to manufacture and will burst along the longitudinal axis.

The solution shown in FIG. 2B can work well if a balloon with the correct compliance is chosen, however wrapping a filament made from polymer or metal around a balloon changes its burst behaviour and will normally cause it to burst circumferentially instead of longitudinally when over-pressurized, creating a serious problem when retrieving the balloon.

### SUMMARY OF THE INVENTION

This invention has a number of aspects including balloons in a variety of embodiments, methods for making such balloons and methods for using such balloons.

The disclosed invention is a medical balloon in which the radial pressure is constant along the cylindrical length of the balloon when inflated. A medical balloon that tends to straighten when inflated will affect the radial force uniformity along the length of the balloon. An inflexible balloon's pressure can go down all the way to zero at the center of the balloon, while the ends exhibit a high pressure against the arterial wall. This feature of the disclosed invention is especially important in certain circumstances, because equal radial pressure along the balloon's axis results in uniform surface contact with the arterial wall. Certain procedures deliver drugs that are coated on medical balloons and benefit from consistent surface contact. While prior art may suffice for the purpose of drug delivery in straight vessels, they are not sufficient in curved vessels such as coronary arteries as the vessel walls may cause non-uniform drug transfer along the length of the medical balloon. The disclosed invention solves this issue as it evenly applies radial pressure to the vessel walls along the entire cylindrical portion of the balloon. This results in uniform or nearly uniform surface contact with the containing vessel. This characteristic of the invention is realized by a balloon that is stretchable in the longitudinal direction but not stretchable (or has lower stretchability) in the circumferential dimension (i.e. it keeps the balloon diameter nearly constant as pressure increases).

A balloon according to an example embodiment has a shallow helical groove formed in the balloon wall. Such a balloon may be designed for use at high inflation pressures (e.g. pressures of at least 5 atm). In some embodiments the balloon is designed for inflation pressures in the range of 10 to 20 atm. The groove increases the bending flexibility of the balloon especially when the balloon is inflated. A filament made from a high tensile strength material with low compliance wraps around the balloon in the embossed groove and is affixed within the groove. The filament prevents the internal pressure from flattening the groove when the balloon is inflated. In some embodiments ends of the balloon are connected together by a flexible member that extends within the balloon.

The walls of medical balloons according to some embodiments are made from non-compliant (or limited compliance) materials. Limited compliance materials that are commonly used today for making balloons (e.g. Nylon 12, PET, PEBAX, polyurethane etc.) are examples of such materials.

The design of balloons according to example embodiments permits the balloons to retain high bending flexibility when inflated to high pressures and yet to maintain a crossing profile at least as good as standard balloons when deflated.

A high pressure balloon is made flexible by having greater compliance in the longitudinal than the circumferential direction which results in a constant radial pressure. The difference in compliance is created by winding a thin metal wire over the balloon and bonding it to the balloon. The wire diameter, strength and winding density are chosen to make the burst rating for a longitudinal split is less than two times that for a circumferential split, in order to have the balloon burst longitudinally in case of over-pressurizing.

Methods according to some embodiments provide ways to manufacture balloons characterized by an axially constant radial pressure using equipment similar or identical to the equipment currently used to make medical balloons. In some embodiments the balloons can have pressure ratings above that of a standard balloon made from the same material and wall thickness.

Further aspects and example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 is a side view of a prior art bent balloon, showing volume change.

FIGS. 2A through 2E illustrate various prior art balloons.

FIG. 3 is a side view of a balloon according to an example embodiment of the invention.

FIG. 4A is a cross section of a wall of a balloon without pressure and FIG. 4B shows the effect of pressure on the balloon wall of FIG. 4A. FIG. 4C shows a possible embodiment which comprises an external sheath or embedded filament.

FIG. 5 is a cross section of a molding tool which may be used to manufacture balloons of the type shown in FIG. 3.

FIG. 6 is a side view of an example balloon incorporating a cord to limit longitudinal expansion.

FIGS. 7A and B are side views of a stent acting as an external structural feature.

FIG. 8A and B are drawings depicting longitudinal and radial forces and the damage caused by each force.

FIG. 9 is a side views of a possible embodiment of the invention.

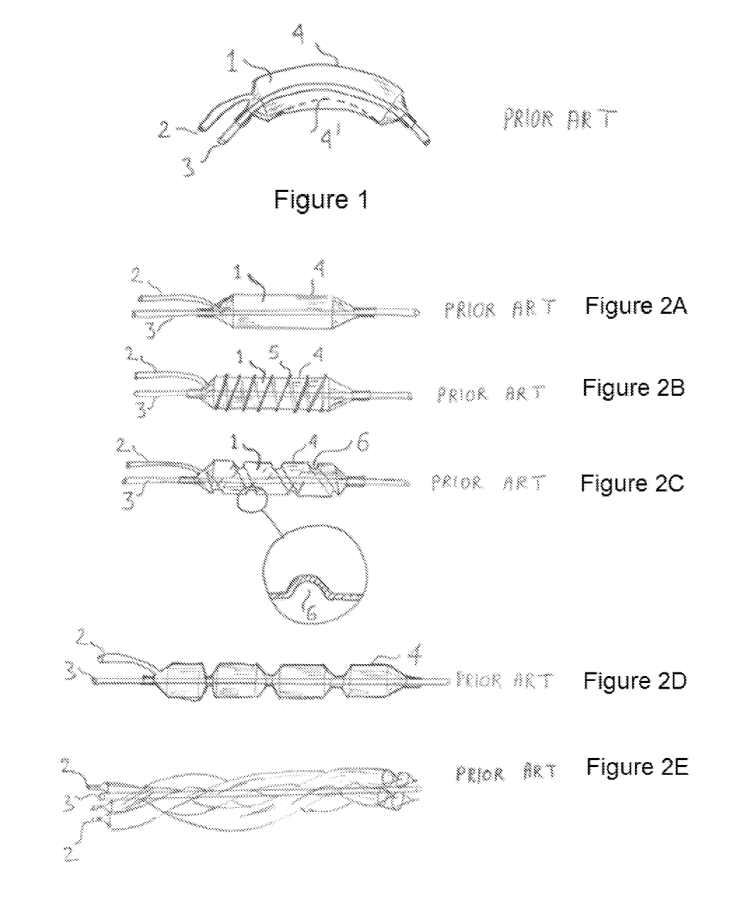

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

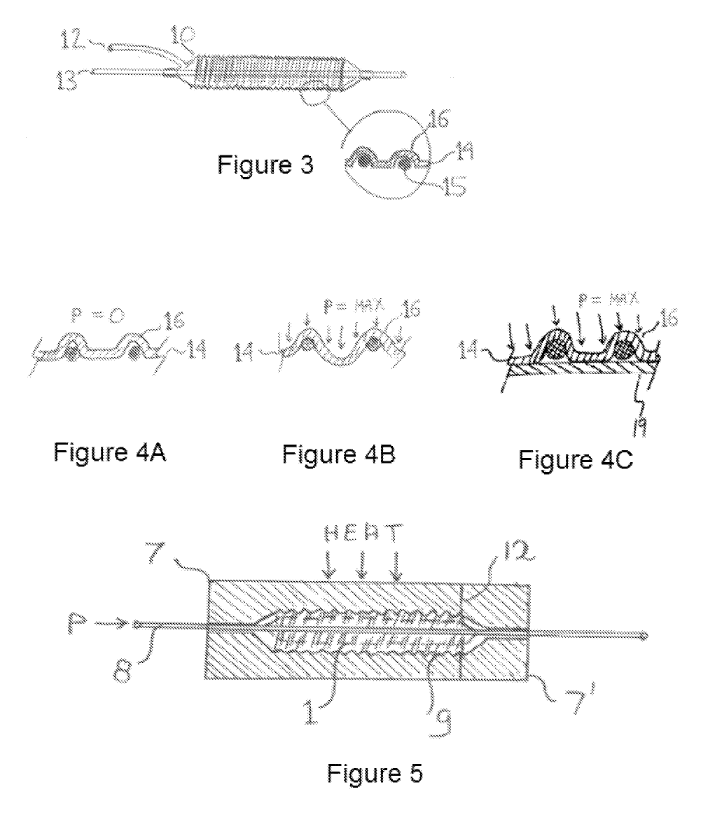

FIG. 3 shows a medical balloon 10 according to an example embodiment. Balloon 10 comprises an inflation tube 12 and optionally incorporates a tube 13 for a guidewire. Balloons according to some embodiments do not incorporate a guidewire tube 13. The wall 14 of balloon 10 is embossed with helical (thread-like) grooves 16 which form a pattern on wall 14. The pitch of grooves 16 may be fixed or may vary along the length of balloon 10. Grooves 16 may comprise a single groove 16 or a plurality of interleaved helical grooves 16 (arranged, for example, like a multi-start thread).

In the illustrated embodiment, grooves 16 cause both the interior surface and the exterior surface of balloon wall 14 to be undulating. In the illustrated embodiment, grooves 16 have depths that are deeper than a thickness of balloon wall 14. The width of the groove is matched to the thickness of the wound filament.

Wall 14 may be made of any suitable material. Example materials for wall 14 include materials such as Nylon12, polyurethane, PET and PEBAX. Wall 14 typically has a thickness in the range of 10 to 30 microns.

A filament 15 made from a high tensile strength material with low compliance is wrapped into groove 16. Filament 15 is affixed within groove 16 in order to prevent separation of filament 15 from wall 14 when balloon 10 is deflated and folded. Filament 15 may, for example, be bonded into groove 16 with a suitable adhesive, embedded in wall 14, or otherwise affixed within groove 16. It is preferable but not mandatory that filament 15 be affixed in groove 16 continuously along its length. In alternative embodiments, filament 15 is affixed in groove 16 at locations that are spaced apart along its length. Filament 15 prevents the pressure inside balloon 10 from flattening groove 16 when balloon 10 is fully inflated.

Filament 15 may comprise a single strand or a plurality of thinner strands. In an example embodiment, filament 15 has a diameter in the range of about 10 microns to about 30 microns. One choice for the material of filament 15 is Kevlar™. For example, filament 15 may be made from a single strand of Kevlar having a diameter in the above range or a plurality of Kevlar strands each having a smaller diameter. Another choice is to use a metal filament, such as type 316 stainless steel. When a metal filament is used it can have a round or rectangular cross section.

Other materials may also be used. For example, if a more diametrically compliant balloon is desired a more compliant material can be used for filament 15 such as monofilament nylon or polyester. Filament 15 can be bonded into groove 16, for example using a suitable adhesive. In one embodiment, bonding of filament 15 is performed by diluting a polyurethane adhesive such as TEXIN™ 5265. An example process that may be used for bonding filament 15 into groove 16 and examples of different suitable adhesives and filament materials are given in U.S. Pat. No. 8,430,846 which is hereby incorporated herein by reference for all purposes.

Typical dimensions of groove 16 are depth of 50 μm to 500 μm and a pitch of 50 μm to 2 mm. In general, as the wall thickness of balloon 10 is increased the dimensions (especially depth) of groove 16 should be increased. Typically, the depth and pitch scale with balloon size, where deeper and coarser grooves are used on larger diameter balloons.

A member that connects ends of balloon 10 to one another may be provided inside balloon 10. In the embodiment illustrated in FIG. 3, such a member is provided by guidewire tube 13. The characteristics of the wall of balloon 10 (wall material, wall thickness and dimensions of groove 16) and the connecting member may be adjusted to achieve a balloon having an axially constant radial pressure. If the balloon wall is too flexible, e. g. the pitch of the wound filament is too small, grooves 16 are too deep or the material of wall 14 is too elastic then the balloon can over bend during pressurization because the connecting member (e.g. guidewire tube 13) which does not stretch much, tends to move towards the inner radius of the curve, which is shorter. This allows the outer radius of the balloon to expand and over bend the balloon. The interaction of the connecting member and the too-flexible balloon wall effectively provides a negative straightening force. If a regular balloon has a strong straightening force and a highly embossed balloon with a wound filament has a negative straightening force, it is possible to create a balloon with an axially constant radial pressure by adjusting the characteristics of the balloon wall. Therefore, the elasticity, embossing pitch and embossing depth can be selected to create a balloon with axially constant radial pressure. There is more than one combination that will result in a nearly axially constant radial pressure balloon. There is an advantage in making the guidewire tube somewhat stretchable, as a balloon also stretches a bit under pressure.

In the embodiment of FIG. 3, grooves 16 facilitate allowing the balloon wall on the inner radius of a bend to compress and the balloon wall on the outer radius of the bend to stretch while keeping the balloon cross-section circular. It is best to use a material that has a larger elastic range than standard balloon materials to allow such compression and stretching.

Filament 15 prevents grooves 16 from disappearing even when balloon 10 is fully inflated. Since the pressure within balloon 10 is pushing out the area between the adjacent turns of wound filament 15, increasing the pressure in the balloon tends to increase the depth of grooves 16 rather than flatten them out. This is illustrated in FIGS. 4A and 4B. FIG. 4A shows the wall of an unpressurized balloon and FIG. 4B shows the balloon wall under full pressure. It has been found that in prototypes of such balloons difference in pressure between the middle section and end of the balloon is near zero when inflated and have axially constant radial pressure at any state of inflation. Note that a possible embodiment of the disclosed invention could further comprise a sheath or external layer 19 to minimize the undulation of the surface, which can be an undesirable trait in certain circumstances, FIG. 4C. Using a ribbon instead of a round filament also helps create a smoother outside surface.

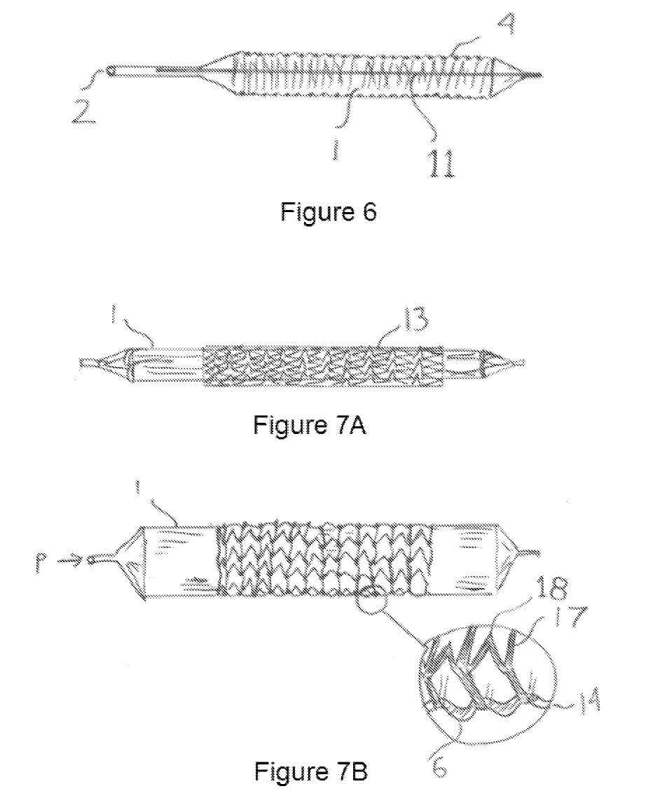

For example, an experimental 6 mm diameter×40 mm long non-compliant balloon made as described herein, with a groove depth and pitch of about 0.4 mm, had over ten times the bending flexibility of a standard balloon (i.e. over 10 times less straightening force) made from the same material and the same thickness. Not only was the radial pressure nearly constant along the length of the balloon, compared to that of a standard balloon, but the crossing profile was also improved and the burst pressure nearly doubled. Since the wound filament increases the burst strength of the balloon, the balloon wall can be made from a softer and more elastic material than a regular balloon, further decreasing the difference in pressure along the length of the balloon. Materials capable of stretching 10% to 50% before breaking are preferred. It was also found that elongation was low, about 10%, and could be eliminated by providing a guidewire tube holding the length nearly constant. For balloons not having a guidewire tube a non-elastic cord can be provided inside the balloon to tie both ends together, as illustrated for example in FIG. 6. Compliance of cord 11 will determine the length compliance of the balloon.

A helical groove 16 may be formed by embossing. Such embossing may be done in conjunction with forming a balloon by methods that are the same as or similar to methods used to manufacture regular balloons. FIG. 5 shows a tube 8 made of a balloon material inserted into a cavity in a mold 7 having a groove pattern 9 machined into its inner wall. Mold 7 is heated, typically to 100-160 degrees C. and tube 8 is expanded into a balloon by pressurizing it. In some embodiments, the balloon is stretched longitudinally at the same time as it is pressurized. After cooling, the balloon can be collapsed by vacuum and removed or the mold can be opened and the balloon removed. Since it is difficult to fabricate the mold from a single piece, a mold split line 12 is provided. For a split line 12 as shown, after cooling, part 7′ is removed and the balloon can be pulled out by using a very slight amount of vacuum or no vacuum at all. If the mold is split longitudinally, the balloon can be removed easily with no need for vacuum but the fit between the mold halves has to be very good in order not to leave a parting line on the finished balloon. Suitable molding machines are commercially available, for example from Interface (www.interfaceusa.com/equipment/balloon-forming-equipment/), and are well known to those of skill in the art. This is one method of manufacturing the disclosed invention, but it is foreseeable than there are many different ways to manufacture a balloon with the desired characteristics.

Using methods of manufacturing such as those described above makes it relatively easy to manufacture balloons that have improved properties relative to conventional balloons.

While preferred embodiments use an embossed groove 16 in the form of a single-start thread, mainly for ease of placing filament 15 into groove 16, it should be understood that any form of groove that results in axially constant radial pressure along the length of the balloon is part of the invention. Examples of possible groove patterns include: isolated rings (which may be parallel to one another), multi-start threads, meshes with a steep angle (i.e. from 60° to)90° relative to the longitudinal axis or any other form.

A different embodiment uses a variable thickness balloon wall to achieve a function like that of a separate filament 15. In such embodiments the balloon is grooved and the balloon wall adjacent to the bottom of each groove is formed to be thicker than parts of the balloon wall between grooves 16. The thickened sections at the bottom of grooves 16 act in the same manner as a filament 15 to prevent grooves 16 from being flattened as the balloon's internal pressure is raised to its maximum working pressure.

Any structural feature preventing the grooved pattern from disappearing when the balloon is pressurized forms part of this invention. For lower pressure balloons it is possible to prevent grooves from disappearing when balloon is pressurized by using a very fine groove pitch and depth. The reason for this is that it takes more force to flatten out a fine pitch grooving pattern than a coarse pitch grooving pattern. For example, the width and depths of grooves may be in the range of 1× to 10× the balloon wall thickness; preferably from 1× to 5× of the wall thickness. For example, where the wall thickness is 30 microns, grooves 16 could have a depth in the range of 30 to 300 microns, preferably 30 to 150 microns.

The following calculation shows how small grooves can be very resistant to being flattened. Assuming the wall thickness is T and the groove radius, measured to the centerline of the thickness is R. Assuming that the groove has a semi-circular cross-sectional shape, the length of the outer layer of the wall from one side of the groove to the other is π(R+0.5 T) and the inner wall layer length is π(R−0.5 T). After flattening the wall the length of either side is πR, and the stress created in either side of the wall is 0.5πT/πR=T/2R.

This simple calculation shows that as the groove radius R becomes smaller and smaller the stresses and forces needed to create them (which are proportional to the stress in the elastic range), become larger and larger. At some combination of groove radius, wall thickness and pressure, the structure of the grooves themselves without a filament 15 will resist collapse at the working pressure of a balloon. For example, a balloon made of 50 micron Nylon, a groove 100 microns wide and 100 micron deep will hold the pressure of about 5 atm.

The invention was tested in simulated artery tissue made from soft silicone, where the arterial conduit curved 90. The simulated tissue contained three sensors that measure the pressure of the balloon on the arterial wall at the center and each end of the balloon. Standard 9.0 mm diameter×40.0 mm long medical balloons, which have little flexibility, displayed high pressure against the tissue on each end of the balloon, but no pressure in the center when inflated. The experiment was repeated with similar balloons that were embossed with grooves with embedded

Kevlar filaments according to disclosed invention. The radial pressure against the simulated tissue wall was constant along the cylindrical axis of the balloon (including the center).

Any part of the balloon that is less compliant than the rest of the balloon can be used to resist flattening of grooves at a working pressure of the balloon. For example, a structural feature can be formed in a wall of uniform thickness by increasing the strength of the material in certain areas. It is well known that irradiating many polymers with ionizing radiation causes cross linking and increases the tensile strength. Irradiating a thread-like helix on the balloon will cause the non-irradiated area to bulge out when balloon is pressurized. The irradiation can be done by a UV laser, X-ray machine or gamma rays. The opposite approach can also be used: weakening a helix like (or ring like) pattern on the balloon by laser ablation or mechanical abrasion will cause this are to bulge out when pressurized. A 248 nm excimer laser is an excellent tool for such controlled material removal. An alternate way is heating the balloon in a helical pattern, using a coiled heater, and stretching it, causing the heated areas to become thinner. In such a case the untreated area forms the structural feature.

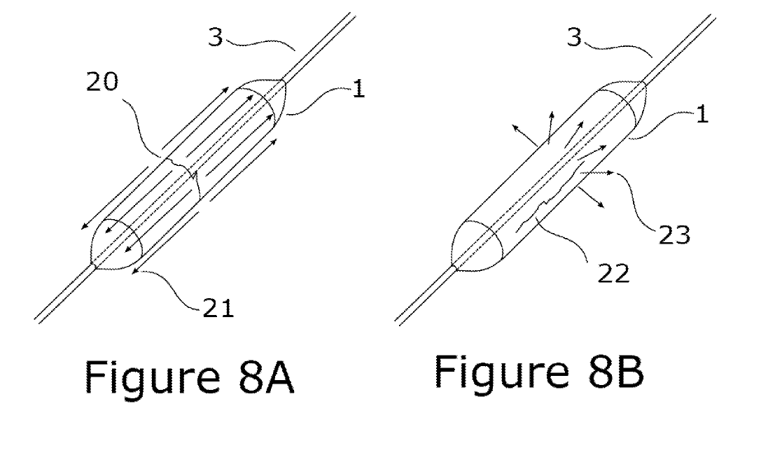

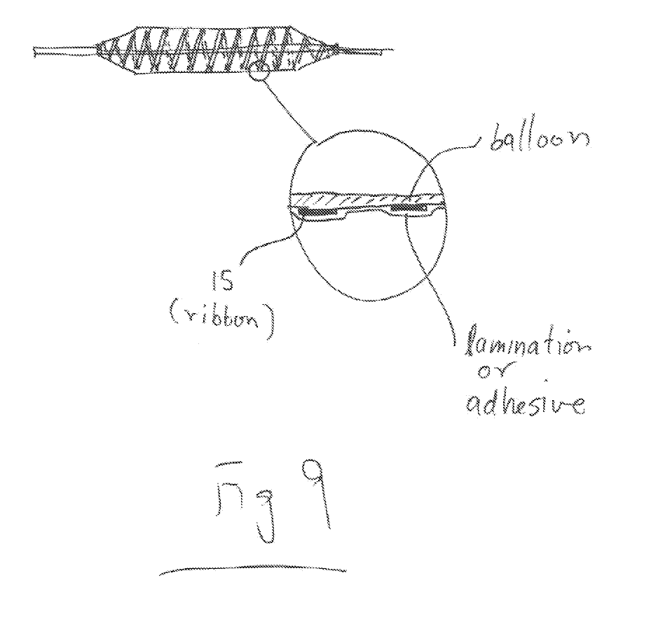

The desired bursting mode of an over-pressurized medical balloon is to split longitudinally rather than along the circumference, for reasons explained earlier. The two modes of bursting are shown in FIG. 8A and 8B, where 8B shows the desired mode (longitudinal) and 8A shows the undesired (circumferential) mode. Longitudinal force 21 and longitudinal bursts 22 should not be confused. Similarly radial force 23 and pressure and radial bursts 20 should not be confused. Radial bursts (along the circumference of the balloon) 20 are caused by longitudinal pressure 21 (FIG. 8A) and longitudinal bursts 22 are caused by radial pressure 23 (FIG. 8B). In a regular balloon with wall thickness T and a diameter D, when pressurized to pressure P, the force trying to break the balloon along the circumference is P πD²/4 while the area of the material resisting this force is πDT. Dividing the two results in the force per unit area, or stress: P πD²/4/πDT=PD/4 T. In the longitudinal direction the force per unit length is PD, while the area of the two walls supporting the force is 2 T, giving a stress of PD/2 T. Because this value is twice the stress that tries to break the balloon along the circumference, the balloon will naturally burst longitudinally. This changes when the balloon is embossed, wound with a filament or both. Referring now to FIG. 3, the area now supporting the balloon against bursting longitudinally increased by the embossing and is further strengthened buy the filament 5. Such a balloon will most likely burst along the circumference unless special measures are taken. The pressure rating for bursting along the circumference did not change, as the filament offers no support in the longitudinal direction. The wire diameter, strength and winding density are chosen to make the burst rating for a longitudinal split is less than two times that for a circumferential split, in order to have the balloon burst longitudinally in case of over-pressurizing.

Since there are at least three different embodiments of making the balloon wall. Each case will be discussed separately.

### Case 1

### Fine Embossing but No Filament

This is similar to FIG. 3 but no filament used in the grooves. Since the stress trying to split the balloon longitudinally is twice the stress trying to split it along the circumference, doubling the wall length by embossing it will make it uncertain where the split would occur. This puts a limit on the depth of the embossing. For full density semi-circular embossing the wall lengthens by a factor of π/2 or about 1.57, thus the balloon will still burst in the desired mode. The same is true for sinusoidal or triangular embossing with depth less than the pitch. For 60 degree triangular embossing. i.e. depth=pitch, profile the ratio is exactly 2. In this case the total length of wall, assuming the embossing has been flattened, cannot exceed twice the wall length of a regular balloon. Recommended material for the balloon is Nylon 12 or PEBAX with a durometer of 65-75 D. Pressurizing the balloon increases the embossing depth, as shown in FIG. 4, but does not change the cross section much as the wall becomes thinner.

### Case 2

Embossing with Filament Inside Groove

This is shown in FIG. 3, the filament typically being fine stainless steel wire. Since the tensile strength of stainless steel is more than ten times that of Nylon, the cross sectional area of the filaments has to be much smaller than that of the balloon wall. A typical balloon wall is about 20 micron, placing a limitation on the winding density of the wire and the pitch of the embossed groove. For small balloons, 2 to 5 mm in diameter, the wire diameter should be around 20 microns and the groove pitch in the range of 0.3-1 mm. Recommended material for the balloon is PEBAX with a durometer of 65-70 D.

### Case 3:

### Self-Formed Embossing, No Embossing or Embedded Filament

The filament, typically metal wire 15, is wound on the balloon and bonded to it. Bonding can be by heating (including passing an electric current through the wire) or by adhesive, typically polyurethane, as shown in Figure

A metal ribbon can be used instead of a round wire for better adhesion and a smoother balloon.

The filament can also be wound in a non-uniform pattern with a decreasing pitch towards the ends of the balloon, as shown in FIG. 9. Such a pattern reduces the tendency of the ends to bulge when deploying a stent, known as “dog-bone” shape. Since the wall area stays the same as a regular balloon the total tensile strength added by the wire should be less than the original tensile strength of the wall. As before, when stainless steel wire is used the cross-sectional area of the wire should be less than 10% of the wall due to the much larger tensile strength of metal wire, typically type 316L stainless steel.

Recommended material for the balloon is PEBAX with a durometer of 60-70D of Polyurethane with a durometer of 55-70D. When such a balloon is pressurized embossing is formed due to the semi-compliant nature of the balloon. For a typical small balloon, using 100 micron×10 μm type 316L stainless ribbon, winding pitch is in the range of 0.3-2mm. For larger balloons a ribbon, such as 10 um×200 um type 316L stainless, is recommended.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

“Elastic modulus” is a characteristic of a material and is a ratio of stress to strain in the material. A material having a smaller elastic modulus is easier to stretch than a material having a higher elastic modulus.

“Elasticity” is a measure of how easily a material can be elastically deformed. The elasticity of a structure such as a balloon wall in a particular direction (e.g. longitudinal or circumferential) is a measure of how much the structure will expand in the indicated direction when placed under tension in that direction.

“Elastic range” means the range through which a material can be stretched without breaking or undergoing a plastic deformation. Elastic range can be expressed as a percentage. for example, if a 10 cm strip of material can be stretched to 13 cm but any more stretching would result in the material undergoing plastic deformation or breaking then the material can be said to have an elastic range of (13−10)/10×100%=30%.

In this disclosure, “compliance” can be understood to have the same meaning as elasticity. A balloon that is considered “compliant” has a larger elastic range that a “non-compliant” balloon.

“High tensile strength” applied to a material means a material that can withstand high forces without breaking or bursting.

“Longitudinal Force” refers to force along the longitudinal axis of the balloon. This should not be confused with a longitudinal bust.

“Radial Force” refers to the force normal to the surface of the balloon. This should not be confused with a radial bust.

“Axially Constant Radial Pressure” refers to constant radial pressure along the longitudinal axis of the balloon.

Where a component (e.g. a tube, wall, assembly, mold, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A method of reducing the straightening force of an inflated medical balloon by making the wall of the balloon having a larger elasticity in the longitudinal direction than in the circumferential direction, wherein said balloon splits longitudinally when over-pressurized.

2. A method as in claim 1, wherein said balloon is forced to split longitudinally by having a balloon wall with different strengths in the radial and longitudinal direction.

3. A method as in claim 1, wherein the strength of the balloon wall in the circumferential direction is less than twice the strength of the longitudinal directions.

4. A method as in claim 1, where lengthening the balloon is longitudinally restricted.

5. A method as in claim 1, wherein said balloon has at least one structural feature to decrease the straightening force of the balloon.

6. A method as in claim 1, wherein said balloon has a wound filament in a helical pattern to control the balloon burst.

7. A method as in claim 1, wherein said balloon has a wound filament in a helical pattern to reduce the straightening force.

8. A method as in claim 1, wherein said balloon has an embossed helical pattern to reduce the straightening force and said helical pattern is reinforced with a wound filament.

9. A method as in claim 1, wherein said balloon maintains constant surface contact with a containing vessel.

10. A medical balloon with a reduced the straightening force, wherein the balloon wall has a larger elasticity in the longitudinal direction than in the circumferential direction and said balloon splits longitudinally when over-pressurized.

11. The medical balloon as in 10, wherein said balloon is forced to split longitudinally by having the balloon wall with different strengths in the radial and longitudinal direction.

12. The medical balloon as in 10, wherein the strength of the balloon wall in the circumferential direction is less than twice the strength of the longitudinal directions.

13. The medical balloon as in 10, wherein the length of the balloon is longitudinally restricted.

14. The medical balloon as in 10, wherein said balloon has at least one structural feature to decrease the straightening force of the balloon.

15. The medical balloon as in 10, wherein said balloon has a wound filament in a helical pattern to control the balloon burst.

16. The medical balloon as in 10, wherein said balloon has a wound filament in a helical pattern to reduce the straightening force.

17. The medical balloon as in 10, wherein said balloon has an embossed helical pattern to reduce the straightening force and said helical pattern is reinforced with a wound filament.

18. The medical balloon as in 10, wherein said balloon maintains constant surface contact with a containing vessel.

19. A medical balloon with axially constant radial pressure, wherein the balloon wall has a larger elasticity in the longitudinal direction than in the circumferential direction and said balloon splits longitudinally when over-pressurized.

20. The medical balloon as in 19, wherein said balloon is forced to split longitudinally by having the balloon wall with different strengths in the radial and longitudinal direction.

21. The medical balloon as in 19, wherein the strength of the balloon wall in the circumferential direction is less than twice the strength of the longitudinal directions.

22. The medical balloon as in 19, wherein the length of the balloon is longitudinally restricted.

23. The medical balloon as in 19, wherein said balloon has at least one structural feature to decrease the straightening force of the balloon.

24. The medical balloon as in 19, wherein said balloon has a wound filament in a helical pattern to control the balloon burst.

25. The medical balloon as in 19, wherein said balloon has a wound filament in a helical pattern to reduce the straightening force.

26. The medical balloon as in 19, wherein said balloon has an embossed helical pattern to reduce the straightening force and said helical pattern is reinforced with a wound filament.

27. The medical balloon as in 19, wherein said balloon maintains constant surface contact with a containing vessel.
